(19) United States
(12) Patent Application Publication
(10) Pub. No.: US 2004/0010311 A1
(43) Pub. Date: Jan. 15, 2004

(54) Customizable integrated prosthetic nipple and areola, processes for the manufacture thereof, and breast prostheses comprising same

(76) Inventors: Lora B. Reynolds, Cartersville, GA (US); Robert J. Halley, Decatur, GA (US); Lisa Marie Hughey, Marietta, GA (US)

Correspondence Address: NEEDLE & ROSENBERG, P.C., SUITE 1000, 999 PEACHTREE STREET, ATLANTA, GA, 30309-3915, US

(21) Appl. No.: 10/196,311

(22) Filed: Jul. 15, 2002

(51) Int. Cl. A61F 2/52

(52) U.S. Cl. 623/7; 623/901

(57) ABSTRACT

The present invention provides an integrated prosthetic nipple and areola suitable for use with a breast prosthesis, wherein the prosthetic nipple and areola comprises a first curable gel zone configured to define a nipple region, and a second curable gel zone integrally connected to the first curable gel zone and configured to define an areola region.

### FIELD OF THE INVENTION

The present invention relates generally to a customizable integrated prosthetic nipple and areola suitable for use with an external breast prosthesis. More specifically, the present invention relates an integrated prosthetic nipple and areola comprised of at least one curable gel, methods for the manufacture thereof, and an external breast prosthesis comprising the same.

### BACKGROUND OF THE INVENTION

A variety of breast prostheses and related accessories are widely known. These prostheses have been developed so that women who have undergone a surgical procedure in which they have had one or possibly both breasts removed, or at least partially removed, for example a mastectomy, may restore not only their cosmetic appearance after surgery, but their self confidence as well. The use of breast prostheses has thus minimized the stigma that may have otherwise been attached to a woman's appearance after breast removal surgery.

To this end, an external breast prosthesis having a realistic nipple and areola region formed as a part of or added thereto is of primary importance to restoring a woman's cosmetic appearance after surgery. As such, several molded rubber prosthetic nipples and areolae have long been available for use in connection with external breast prostheses. However, these prosthetic nipples and areolae have traditionally been unrealistic in appearance and feel, oftentimes hard, and inconvenient by requiring the application of an adhesive in order to adhere same to the external breast prosthesis.

Moreover, in an effort to eliminate the need for adhesives, several attempts have been made at incorporating a prosthetic nipple and areola into a film encapsulated breast prosthesis whereby the nipple and areola are encapsulated within the film layers of the prosthesis itself, such as that disclosed in U.S. Pat. No. 5,370,688 to Shulz, et al. However, these nipples and areolae are still made from molded rubber and once again present an unrealistic appearance and feel, and remain quite hard to the touch. In addition, the use of an encapsulating inner film to position the nipple and areola in the front of the breast prosthesis has similarly imparted a nonrealistic circular pattern to the nipple and areola.

Accordingly, it is an object of the present invention to provide an improved and more realistic prosthetic nipple and areola having a diffuse edge and the capability to be customized to the individual wearer through color matching, size, and variations in texture and firmness.

### SUMMARY OF THE INVENTION

Among other aspects, the present invention provides an improved prosthetic nipple and areola suitable for use with breast prostheses. More specifically, the prosthetic nipple and areola is comprised of one or more curable gel layers that can be positioned on or within a breast prosthesis. Furthermore, the prosthetic nipple and areola can additionally be customized to the individual wearer for size, shape, color, firmness, and/or texture.

In a first embodiment, the invention comprises an integrated prosthetic nipple and areola suitable for use with a breast prosthesis. The prosthetic nipple and areola comprise a first curable gel zone configured to define a nipple region, and a second curable gel zone integrally connected to the first curable gel zone and configured to define an areola region. The first and second curable gel zones may each be comprised of a silicone gel. Additionally, the first and the second curable gel zones may be formed as one homogenous zone of a curable gel. The first curable gel zone may be comprised of more than one layer of curable gel, as may the second curable gel zone. Also, the areola of the prosthetic nipple may have a diffuse edge.

The invention therefore also comprises a breast prosthesis comprising the integrated and above-described prosthetic nipple and areola. Accordingly, the invention comprises a breast prosthesis comprised as a two layer film encapsulated gel filled prosthesis comprising a first inner layer and a second outer layer, respectively, each comprised of a curable elastic material region, and an integrated prosthetic nipple and areola comprised of a first curable gel zone configured to define a nipple region, and a second curable gel zone integrally connected to the first curable gel zone and configured to define an areola region.

Yet another embodiment of the invention teaches a process for the manufacture of an integrated prosthetic nipple and areola suitable for use in a breast prosthesis, comprising the steps of providing a film formed into a mold, the mold having a surface design configured to define an integrated prosthetic nipple and areola having a desired shape and size; depositing at least one first curable gel layer into the mold to provide a first curable gel zone to define the nipple region; depositing a least one second curable gel layer into the mold to provide a second curable gel zone to define the areola region in direct communication with the nipple region; and heat curing the first and second curable gel zones, the respective gel zones each comprising a silicone gel.

The invention also teaches a process for the manufacture of a film encapsulated gel filled breast prosthesis comprising an integrated prosthetic nipple and areola, the process comprising the steps of providing a film envelope configured to define an interior volume, the film envelop comprising a fill opening and wherein the film envelope at least partially encapsulates the prosthetic nipple and areola; at least partially filling the interior volume of the film envelope by passing a curable elastic material through the fill opening; and sealing the fill opening.

In still another inventive process, the invention teaches a process for the manufacture of a two-layer film encapsulated gel filled breast prosthesis comprising an integrated prosthetic nipple and areola, comprising the steps of providing a first film envelope configured to define a first interior volume; providing a second film envelope joined to the first film envelope along a common side edge to thereby define a second interior volume, wherein the second film envelope at least partially encapsulates the prosthetic nipple and areola, and wherein the second film envelope and the first film envelope share a common interstitial film wall, the first and the second film envelopes further comprising a respective first and a second fill opening extending from the common side edge of the respective first and second film envelopes to the respective first and second interior volumes; at least partially filling the second interior volume by passing a curable elastic material precursor through the second fill opening; at least partially filling the first interior volume by passing a self-shaping dispersion through the first fill opening; and sealing the first and second fill openings.

Additional advantages and embodiments of the invention will be obvious from the description, or may be learned by practice of the invention. Further advantages of the invention will also be realized and attained by means of the elements and combinations particularly pointed out in the appended claims. Thus, it is to be understood that both the foregoing general description and the following detailed description are exemplary and explanatory of certain embodiments of the invention, only, and are not otherwise restrictive of the invention as claimed.

### BRIEF DESCRIPTION OF THE DRAWINGS

FIG. 1 is a schematic cross-sectioned side elevational view of a first embodiment of a prosthetic nipple and areola of the invention.

FIG. 2 is a top plan view of the prosthetic nipple and areola of FIG. 1.

FIG. 3 is a schematic cross-sectioned side elevational view of a second embodiment of the prosthetic nipple and areola of the invention.

FIG. 4 is a schematic cross-sectioned side elevational view of a third embodiment of the prosthetic nipple and areola of the invention.

FIG. 5 a schematic cross-sectioned side elevational view of a first embodiment of a breast prosthesis in combination with the prosthetic nipple and areola of FIG. 1.

FIG. 6 a schematic cross-sectioned side elevational view of a second embodiment of a breast prosthesis in combination with the prosthetic nipple and areola of FIG. 1.

### DETAILED DESCRIPTION

The present invention may be understood more readily by reference to the following detailed description and any examples provided herein. The terminology used herein is used only for the purpose of describing particular embodiments of the present invention and is not intended to be limiting in any way. Furthermore, it must also be noted that as used in the specification and the appended claims, the singular forms “a,” “an,” and “the” comprise plural referents unless the context clearly indicates otherwise. For example, reference to a component in the singular is intended to comprise a plurality of components.

Ranges may be expressed herein as from “about” or “approximately” one particular value and/or to “about” or “approximately” another particular value. When such a range is expressed, another embodiment comprises from the one particular value and/or to the other particular value. Similarly, when values are expressed as approximations, by use of the antecedent “about” or “approximately”, it will be understood that the particular value forms another embodiment. Moreover, it should be understood that throughout this application, where publications are referenced, the disclosures of these publications in their entireties are hereby incorporated by reference into this application in order to more fully describe the state of the art to which this invention pertains.

As used herein: the term “zone” is intended to refer to an area of the prosthesis distinguished from adjacent parts by its distinctive features or characteristics, including such features as color, size, shape, contents, and stiffness or firmness; the term “nipple” is intended to refer to an outward projection, formed or otherwise defined, near the center of a human mammary gland, and any of various prosthetic devices functioning like or intended to resemble the same; the term “areola” is intended to refer to the ring shaped area of colored tissue located about the nipple of a human breast or external breast prosthesis as defined above; the term “integrated” is intended to refer to adjacent parts or zones, as defined above, that are in direct communication or contact with each other such that together they are unified as one larger, indistinguishable, unit; and the term “suitable” is intended to refer to the ability of a particular element, feature, property, article, process and the like to perform the designated function.

To this end, it should be understood that by virtue of this “suitability” or ability to perform the designated function, it does not necessarily follow that the particular element, feature, property, article, process and the like must actually be used or perform as such. For example, as used herein, a prosthetic nipple and areola that is suitable for use in an external breast prosthesis is intended to refer to a nipple and areola that is capable of being used in the suggested manner, but is not limited to such use. That is to say, the prosthetic nipple and areola can also exist as a stand-alone article, independent from an external breast prosthesis.

As used herein, the term “optional” or “optionally” means that the subsequently described event or circumstance may or may not occur, and that the description includes instances where any such event or circumstance occurs, and instances where it does not. For example, the phrase “optionally substituted lower alkyl” means that the lower alkyl group may or may not be substituted and that the description includes both unsubstituted lower alkyl and lower alkyl where there is substitution.

As used herein, the term “diffuse edge” is intended to refer to an edge between two zones, regions and/or layers of cured gel that is not sharp or clearly delineated, such that there is a gradual color change between the two zones, regions and/or layers. The diffuse edge will also exhibit a slightly irregular shape, or border between the two zones.

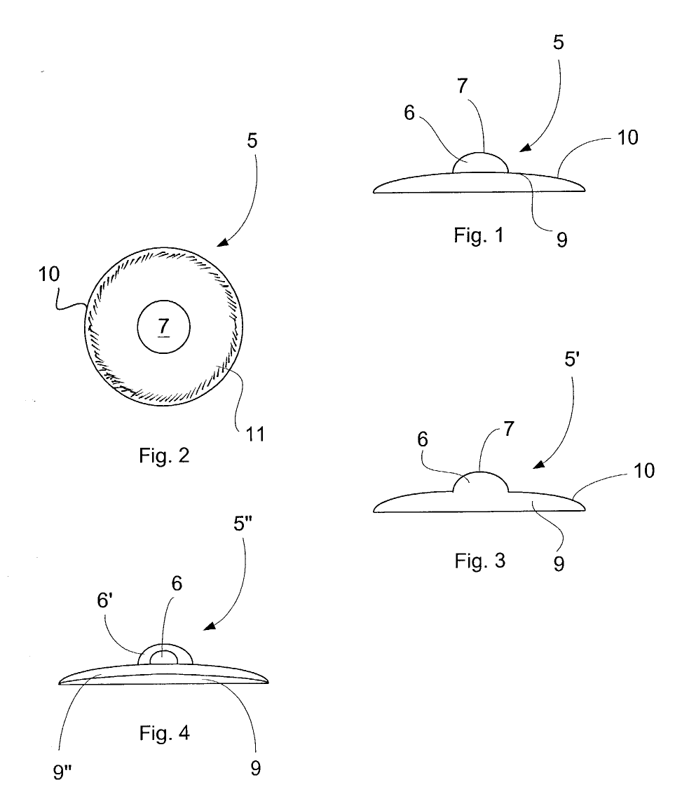

As indicated above, in a first aspect, the present invention provides a customizable integrated prosthetic nipple and areola suitable for use with an external breast prosthesis. In accordance with this aspect, and referring now to the drawings, in which like reference numerals indicate like parts throughout the several views, a first embodiment of a prosthetic nipple 5 is shown in FIGS. 1 and 2. The nipple is formed of a first curable gel zone 6 forming a nipple region 7, and a second curable gel zone 9 forming an areola region 10 of the prosthetic device. A diffuse edge region 11 circumscribes the periphery of, and is defined within, the areola region 10. It is anticipated that each of the nipple and areola regions, and more particularly the curable gels forming such regions, may be suitably and separately dyed, tinted, or colored to represent a human nipple and areola in exterior appearance.

Accordingly, the curable gels can further comprise one or more additives such as colorants, pigments, polymer particles, fibers, fillers, density reducing agents or materials, which allow additional customization of the prosthesis. To this end, in a preferred embodiment, one or more desired pigments and/or colorants are added to the curable gel in an amount ranging from approximately 0.02 wt. % to approximately 0.5 wt. % based on the total weight percent of the particular curable gel.

The respective first and second gel zones are each comprised of a curable gel and/or an elastic material precursor. Once cured, the first and the second curable gel zones constitute a relatively firm elastic material. To this end, the relatively firm elastic material is capable of maintaining the desired shape of the nipple and/or areola region and preventing wrinkling while at the same time remaining sufficiently soft to yield to the touch in a realistic and responsive manner.

Suitable curable gels for use in the disclosed embodiments of the present invention include, without limitation, curable thermoplastic and/or thermoset materials, such as curable silicone gels and curable polyurethane gels.

In order to provide the capability to customize the nipple and areola to the individual wearer, it will be appreciated that the respective first and the second curable gel zones can each comprise varying amounts of a curable gel or gels to adjust for the desired size. Toward this end, it is anticipated that one of ordinary skill in the art would readily be able to optimize such amount(s) for the individual wearer. Nonetheless, in a preferred embodiment, the first curable gel zone and the second curable gel zone each comprise a gel in the range of from approximately 1.0 mL to approximately 5.0 mL of one or more curable gels; including, without limitation, such amounts as 1.5, 2.0, 2.5, 3.0, 3.5, 4.0 and 4.5 mL.

It will also be appreciated that the relative firmness of the cured gel material can also be selected as desired to attain the advantages described hereinabove, e.g., a realistic texture, sufficiently soft to the touch, and an ability to maintain a desired shape without wrinkling. To that end, the cured gel will preferably have a firmness, measured as a penetration value in the range of from approximately 100 to approximately 300, including such values as 125, 150, 175, 200, 225, 250 and 275. Such values represent measurements made with a PNR 6 penetrometer, manufactured by FA Dargatz.

Although the prosthetic nipple and areola of the present invention have been defined as comprising a first curable gel zone and a second curable gel zone respectively, it is understood that first and the second curable gel zones can each comprise one or more different curable gels or, alternatively, each zone can be comprised of the same curable gel. To this end, and as illustrated in FIG. 3, in one embodiment, the first curable gel zone 6 and the second curable gel zone 9 together form one homogenous and contiguous curable gel zone comprising any one or more of the suitable curable gels defined above. Alternatively, in another embodiment, the first curable gel zone is comprised of at least one curable gel not present in the second curable gel zone. In a preferred embodiment, therefore, the first curable gel zone configured to define a nipple region is comprised of a curable silicone gel. In another preferred embodiment, the second curable gel zone, configured to define an areola region, is comprised of a curable polyurethane gel.

Preferably, the nipple zone and the areola zone respectively, each comprises one layer of curable gel. However, it should also be understood that each curable gel zone can further comprise one or more layers of a curable gel or gels. To this end, and as illustrated in FIG. 4, it will be appreciated that the presence of one or more layers of the first curable gel 6, 6′ and the second curable gel 9, 9′, respectively, further enhances the customization of the prosthesis by enabling the prosthetic nipple and/or areola region to comprise several variations in color, density, stiffness and penetration and the like. As such, the resulting prosthesis will desirably replicate a more realistic nipple and areola.

According to the invention, each layer of curable gel is preferably deposited in a thickness ranging from approximately 0.5 mm to approximately 3.5 mm, including such values as 1.0, 1.5, 2.0, 2.5 and 3.0 mm. It is understood, however, that the thickness can of course vary below or above the stated ranges depending on the size of nipple and areola desired.

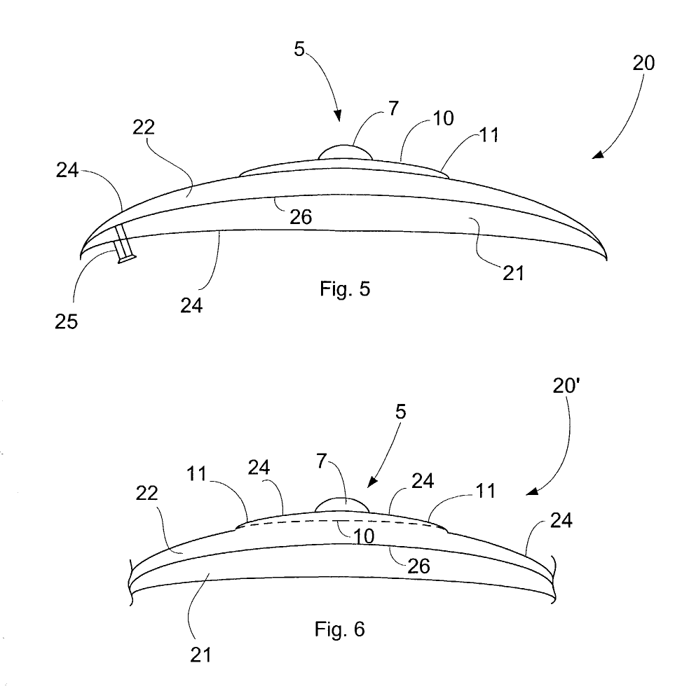

In yet another aspect, the present invention further provides an external breast prosthesis containing the integrated prosthetic nipple and areola as described above, and as illustrated in FIGS. 5 and 6.

Although the prosthetic nipple and areola of the present invention is suitable for use with virtually any form of an external breast prosthesis, in a preferred embodiment, an encapsulated gel filled breast prosthesis 20 is formed of one or more layers. As shown in FIG. 5, the breast prosthesis has a first or inner layer 21 and a second or outer layer 22, the layers being formed by a suitable film 24 adapted for being shaped or otherwise formed to receive the constituent material of the respective layers of the prosthesis therein, and to form the completed device, as known. A conventional fill valve 25, which may comprise a bifurcated fill valve, is provided for filling the layer, or layers, comprising the prosthetic breast.

Accordingly, the integrated prosthetic nipple and areola of the present invention can be used with any one of the breast prosthesis disclosed in U.S. Pat. Nos. 4,172,298, 4,249,975, and 4,247,351, directed to single layer prostheses; as disclosed in U.S. Pat. Nos. 4,950, 291, and 5,922,023 directed to two layer breast prostheses; and to U.S. Pat. Nos. 5,352,307, 5,792,292, and 5,738,812 which describe prostheses with various other features. Toward that end, the entire disclosures of the aforementioned references are hereby incorporated by reference in their entireties herein for all purposes.

Furthermore, in a preferred embodiment, the integrated nipple and areola of the present invention is suitable for use with a two-layer external breast prosthesis having a self shaping feature, such as that disclosed in EP 0768068B1, and U.S. patent application Ser. No. 10/081,116, the entire disclosures of which are incorporated herein by reference in their entirety for all purposes.

It will be appreciated that the preparation of the relatively firm silicone gel based elastic material forming the outer layer of the prosthesis is generally within the expertise of those skilled in the art. An appropriate silicone rubber is described in detail by Patzke and Wohlfarth in their article entitled “Venetzungssysteme beim Siliconkautschuk,” published in the CHEMIKER-ZEITUNG 97^{th} year (1973) No.4, pages 176-180. Furthermore, the relative stiffness of the silicone gel material may be selected as desired to attain the advantages described hereinabove. Preferably, the silicone rubber material forming the outer layer will have a penetration measured in a range from 20.0 to 24.0 mm. Such values represent measurements with a “precision” penetrometer using a 15 gram cone having an aluminum tip with a height of 0.6 inches, a base diameter of 0.33 inches, and a plastic cone body extending 1.13 inches from the tip base and having a base diameter of 2.56 inches.

In accordance with this particular embodiment of a breast prosthesis, and other two-layer breast prostheses within the scope of the present invention, the first inner layer and second outer layer of the two layer breast prosthesis are each confined within a first film envelope and a second film envelope (FIGS. 5 and 6), respectively. Preferably, the respective first and second film envelopes share a common, interstitial film wall 26 therebetween. Therefore, the respective inner and outer layers of the two-layer prosthesis are preferably separated by this interstitial film wall. To this end, any suitable film composition known to one of ordinary skill in the art can be used with the present invention. However, in a preferred embodiment, the films used to form the respective film envelopes are comprised of a polyurethane film.

Likewise, in accordance with this aspect of the present invention, the prosthetic nipple and areola is at least partially encapsulated by the film envelope of the second, outer layer, such that the nipple and areola portion is adhered to and/or positioned in the desired location of the breast prosthesis. To this end, the prosthetic nipple and areola are preferably not separated from the cured or curable elastic material within the second, outer layer by an interstitial film layer, as illustrated in FIG. 6. This advantageously enables the border of the areola region to exhibit a diffused edge, as described above, rather than an unnatural circular pattern which typically results when the nipple and areola are separated from the remainder of the form via an interstitial film.

Alternatively, the present invention thus provides a two-layer breast prosthesis 20, 20′ comprising a first film envelope configured to define a first interior volume or layer 21, and a second film envelope joined to the first film envelope along a common side edge 26 to thereby define a second interior volume or layer 22, wherein the second film envelope and the first film envelope share a common film wall. A first and a second sealed fill opening 25 (FIG. 5), respectively, which in each embodiment of the invention can comprise a bifurcated fill valve or opening having separate channels passing to each respective body portion or layer of the prosthesis, each extending from the common side edge of the first film envelope and the second film envelope to the respective first and second interior volumes. A first material is placed within the first film envelope forming an interior body portion of the prosthesis, where the first material may comprise, for example, a self-shaping material. A second material is placed within the second film envelope to form an exterior body portion of the prosthesis, the second material comprising an elastic material and/or curable elastic material precursor.

Once again, in accordance with this embodiment (FIG. 6), the prosthetic nipple and areola may be at least partially encapsulated by the film envelope of the second, outer layer, such that the nipple and areola portion is adhered to and/or positioned in the desired location of the breast prosthesis. Also, the prosthetic nipple and areola is again preferably not separated from the cured or curable elastic material within the second, outer layer, by an inner film layer, thus enabling the border of the areola region to exhibit the diffused edge.

In yet another aspect, the present invention provides a method for the manufacture of the prosthetic nipple and areola as described above. Accordingly, in one embodiment, the process comprises the steps of providing a film, vacuum formed into a mold, wherein the mold has a surface design configured to define an integrated prosthetic nipple and areola having a desired shape and size. Then depositing at least one first curable gel layer into the mold to provide a first curable gel zone configured to define a nipple region and depositing at least one second curable gel layer into the mold to provide a second curable gel zone configured to define and areola region that is in communication with the said nipple region. After the deposition of curable gel is complete, the first curable gel zone and the second curable gel zone are each heat cured.

As indicated above, the first and second curable gel layers can be deposited as a series of layers or as one homogenous layer of curable gel.

The first and second curable gel zones can be cured through infra-red, radiant, heated mold, conductive and/or convective heat processes, as known. To this end, it will be appreciated that the requisite processing conditions, e.g., temperature and duration of curing, will of course vary depending on the particular amounts and types of curable gel or gels selected. However, such optimum conditions will be readily obtained by one of ordinary skill in the art through no more than routine experimentation. Nonetheless, in a preferred embodiment, the curable gels are heat cured. To this end, the gels are preferably heat cured using infra-red or heated mold processes whereby the gel is heated to a temperature in the range of from approximately 150° C. to approximately 250° C., including such temperatures as 160° C., 170° C., 180° C., 190° C., 200° C., 210° C., 220° C., 230° C., and 240° C. Of course, it is understood that the required temperature will be dependent on the particular gel and amount used. However, such conditions will be readily obtained by one of ordinary skill in the art through no more than routine experimentation.

As described at length above, the integrated prosthetic nipple and areola of the present invention is suitable for use in an external breast prosthesis. As such, it follows that the scope of present invention further provides a process for the manufacture of an external breast prosthesis comprising the prosthetic nipple and areola of the present invention.

To this end, in one embodiment, the present invention provides a process for the manufacture of a film encapsulated gel filled breast prosthesis comprising the steps of providing a film envelope configured to define an interior volume, wherein the film envelope comprises a fill opening and wherein the film envelope at least partially encapsulates the prosthetic nipple and areola of the present invention. The interior volume of the film envelope is at least partially filled by passing a curable gel or elastic material through the fill opening followed by the sealing of the fill opening. Although any means suitable for sealing the film opening can be used with the present invention, it is preferred that the film opening be heat sealed.

Accordingly, the at least partially filled film envelope is then heat treated in a mold having a surface design configured to define a desired shape and size of an external breast prosthesis to thereby cure the gel or elastic material and to provide a breast prosthesis having the desired shape and size. Once again, the heat curing step can be performed via infra-red, radiant, heated mold, conductive, or convective heat processes or any combination thereof.

Alternately, the present invention further provides a process for the manufacture of a two-layer film encapsulated gel filled breast prosthesis comprising the integrated prosthetic nipple and areola of the present invention. Accordingly, the process comprises the steps of providing a first film envelope configured to define a first interior volume; providing a second film envelope joined to the first film envelope along a common side edge to thereby define a second interior volume wherein the second film envelope at least partially encapsulates a prosthetic nipple and areola a described herein, and wherein the second film envelope and the first film envelope further share a common interstitial film wall, the first and the second film envelopes further comprising a respective first and a second fill opening extending from the common side edge of the respective first and second film envelopes to the respective first and second interior volumes.

The second interior volume is at least partially filled by passing a curable gel or elastic material precursor through the second fill opening and the first interior volume is at least partially filled by passing a self-shaping dispersion through the first fill opening. After the first and the second interior volumes are at least partially filled, the first and second fill openings are then sealed. Although any means suitable for sealing the film openings can be used with the present invention, it is preferred that the film openings be heat sealed.

According to this embodiment, the at least partially filled film envelopes are then heat treated in a mold having a surface design configured to define a desired shape and size of an external breast prosthesis to thereby cure the gel or elastic material and to provide a breast prosthesis having the desired shape and size. Once again, the heat curing step can be performed via infra-red, radiant, heated mold, conductive, or convective heat processes or any combination thereof.

It should also be understood that the integrated prosthetic nipple and areola of the present invention can first be formed inside of an existing film envelope or, alternatively, the film envelope can be manufactured subsequent to the formation of the prosthetic nipple and areola, as previously described herein, by sealably affixing a second film to the film previously used in the manufacture of the prosthetic nipple and areola. To this end, although optional, it may be desired to first coat the back side of the prosthetic nipple and areola with a layer of curable silicone in order to prevent the second film from adhering directly to the back of the prosthetic nipple and areola during the process of sealably affixing the second film to the film already containing the nipple and areola.

Although several embodiments of the invention have been disclosed in the foregoing specification, it is understood by those skilled in the art that many modifications and other embodiments of the invention will come to mind to which the invention pertains, having the benefit of the teaching presented in the foregoing description and associated drawings. It is thus understood that the invention is not limited to the specific embodiments disclosed hereinabove, but rather is intended to cover such alternatives, modifications, and equivalents as may be included within the spirit and scope of the invention as defined by the appended claims. To this end, one skilled in the art will appreciate that in practicing the present invention, only reasonable and routine experimentation will be required to optimize such variables and conditions as set forth herein.

We claim:

1. An integrated prosthetic nipple and areola suitable for use with a breast prosthesis, said prosthetic nipple and areola comprising:
a first curable gel zone configured to define a nipple region; and
a second curable gel zone integrally connected to the first curable gel zone and configured to define an areola region.

2. The prosthetic nipple and areola of claim 1, wherein the first curable gel zone is comprised of a silicone gel.

3. The prosthetic nipple and areola of claim 1, wherein the second curable gel zone is comprised of a silicone gel.

4. The prosthetic nipple and areola of claim 1, wherein the first curable gel zone comprises in the range of from approximately 1.0 mL to approximately 5.0 mL of a curable gel.

5. The prosthetic nipple and areola of claim 1, wherein the second curable gel zone comprises in the range of from approximately 1.0 mL to approximately 5.0 mL of a curable gel.

6. The prosthetic nipple and areola of claim 1, wherein the first and the second curable gel zones form one homogenous zone of curable gel.

7. The prosthetic nipple and areola of claim 1, wherein the first curable gel zone comprises more than one layer of curable gel.

8. The prosthetic nipple and areola of claim 1, wherein the second curable gel zone comprises more than one layer of curable gel.

9. The prosthetic nipple and areola of claim 1, wherein the first and the second curable gel zones are heat cured.

10. The prosthetic nipple and areola of claim 1, wherein the areola has a diffuse edge.

11. A breast prosthesis comprising the integrated prosthetic nipple and areola of claim 1.

12. The breast prosthesis of claim 11, wherein the breast prosthesis comprises a film encapsulated gel filled layer.

13. The breast prosthesis of claim 11, wherein the breast prosthesis comprises a two layer film encapsulated gel filled prosthesis comprising:
a first inner layer; and
a second outer layer comprised of:
i) a curable elastic material region, and
ii) an integrated prosthetic nipple and areola comprising:
a first curable gel zone configured to define a nipple region; and
a second curable gel zone integrally connected to the first curable gel zone and configured to define an areola region.

14. The prosthesis of claim 13, wherein the first inner layer is comprised of a self shaping dispersion.

15. The prosthesis of claim 13, wherein the first inner layer is comprised of a curable elastic material.

16. The breast prosthesis of claim 13, wherein the integrated prosthetic nipple and areola is not separated from the curable elastic material region by an interstitial layer of film.

17. The breast prosthesis of claim 11, wherein the areola region has a diffuse edge.

18. A process for the manufacture of an integrated prosthetic nipple and areola suitable for use in a breast prosthesis, comprising the steps of:
providing a film formed into a mold, wherein the mold has a surface design configured to define an integrated prosthetic nipple and areola having a desired shape and size;
depositing at least one first curable gel layer into the mold to provide a first curable gel zone configured to define a nipple region;
depositing a least one second curable gel layer into the mold to provide a second curable gel zone configured to define an areola region in direct communication with said nipple region; and
heat curing the first curable gel zone and the second curable gel zone.

19. The process of claim 18, wherein the film is formed into a mold under vacuum.

20. The process of claim 18, wherein the at least one first curable gel layer is comprised of a silicone gel.

21. The process of claim 18, wherein the at least one second curable gel layer is comprised of a silicone gel.

22. The process of claim 18, wherein the first curable gel zone comprises in the range of from approximately 1.0 mL to approximately 5.0 mL of curable gel.

23. The process of claim 18, wherein the second curable gel zone comprises in the range of from approximately 1.0 mL to approximately 5.0 mL of curable gel.

24. The process of claim 18, wherein the first curable gel zone and the second curable gel zone are cured by infra-red heat, radiant heat, conductive heat, convective heat, or any combination thereof.

25. A process for the manufacture of a film encapsulated gel filled breast prosthesis comprising an integrated prosthetic nipple and areola, comprising the steps of:
providing a film envelope configured to define an interior volume, wherein the film envelope comprises a fill opening and wherein the film envelope at least partially encapsulates the prosthetic nipple and areola;
at least partially filling the interior volume of the film envelope by passing a curable elastic material through the fill opening; and
sealing the fill opening.

26. The process of claim 25, wherein the fill opening is heat sealed.

27. The process of claim 25, further comprising:
heat treating the at least partially filled film envelope in a mold having a surface design configured to define a desired shape and size of a breast prosthesis to thereby cure the elastic material and to provide a breast prosthesis having the desired shape and size.

28. The process of claim 27, wherein the heat treating comprises the use of infra-red heat, radiant heat, conductive heat, convective heat, or any combination thereof.

29. A process for the manufacture of a two-layer film encapsulated gel filled breast prosthesis comprising an integrated prosthetic nipple and areola, comprising the steps of:
a) providing a first film envelope configured to define a first interior volume;
b) providing a second film envelope joined to the first film envelope along a common side edge to thereby define a second interior volume, wherein the second film envelope at least partially encapsulates a prosthetic nipple and areola therein, wherein the second film envelope and the first film envelope share a common interstitial film wall, and wherein the first and the second film envelopes further comprise a respective first and second fill opening extending from the common side edge of the respective first and second film envelopes to the respective first and second interior volumes;
c) at least partially filling the first interior volume of step a) by passing a self-shaping dispersion through the first fill opening;
d) at least partially filling the second interior volume of step b) by passing a curable elastic material precursor through the second fill opening; and
sealing the first and second fill openings.

30. The process of claim 29, wherein the first and the second fill openings are heat sealed.

31. The process of claim 29, further comprising:
heat treating the at least partially filled joined first film envelope and second film envelope in a mold having a surface design configured to define a desired shape and size of a breast prosthesis to thereby cure the elastic material in the second film envelop and to provide a breast prosthesis having the desired shape and size.

32. The process of claim 31, wherein the heat treating comprises the use of infra-red heat, radiant heat, conductive heat, convective heat, or any combination thereof.
